(19) United States
(12) Patent Application Publication
(10) Pub. No.: US 2021/0299304 A1
(43) Pub. Date: Sep. 30, 2021

(54) STERILIZATION SYSTEMS USING UV LIGHT

(71) Applicant: C. Cretors & Company, Wood Dale, IL (US)

(72) Inventors: Patrick John Concannon, Chicago, IL (US); Nenad Vidojevic, Chesterton, IN (US); Andrew Cretors, Wood Dale, IL (US)

(21) Appl. No.: 17/219,159

(22) Filed: Mar. 31, 2021

Related U.S. Application Data
(60) Provisional application No. 63/003,049, filed on Mar. 31, 2020.

(51) Int. Cl. A61L 2/10; A61L 2/26

(52) U.S. Cl. CPC A61L 2/10; A61L 2/26; A61L 2202/26; A61L 2202/11; A61L 2202/122; A61L 2202/121; A61L 2202/24; A61L 2202/14

(57) ABSTRACT

Systems and methods for sterilizing items, such as respirators, masks, and other personal protection equipment, using UV-C light are disclosed herein. In some embodiments, counter-top systems for providing 360-degree germicidal irradiation of respirators and other items are disclosed herein. In some embodiments, the systems include one or more UV-C lamps positioned inside of a rotatable structure that supports items for sterilization, and one or more UV-C lamps positioned outside of the rotatable support structure.

### CROSS-REFERENCE TO RELATED APPLICATION

This application claims the benefit of U.S. Provisional Patent Application No. 63/003,049, filed Mar. 31, 2020, and titled “STERILIZATION SYSTEMS USING UV LIGHT,” which is incorporated herein by reference in its entirety.

### TECHNICAL FIELD

The present disclosure is generally related to systems and methods that use UV light to sterilize items, such as safety goggles, keys, fobs, office supplies, portable electronic devices, and so on.

### BACKGROUND

UV-C (which can also be referred to as UVC or ultraviolet C) refers to radiation that is in the region of the ultraviolet spectrum and extends from about 200 to about 280 nanometers (nm) in wavelength. The use of UV-C light to inactivate human pathogens including, for example, coronavirus, is well-known. In some situations, it may be necessary or desirable to sterilize various items. Conventional systems for disinfecting items with UV-C light use lamps that shine on the items. Because UV-C light does not reflect much, such systems are often unsuitable for items having three-dimensional or non-simple shapes, because the light cannot reach all the surfaces of the item. Accordingly, systems and methods are needed that can facilitate efficient sterilization of items.

### BRIEF DESCRIPTION OF THE DRAWINGS

FIG. 1A is a partially exploded isometric view of a UV-C light sterilization system configured in accordance with embodiments of the present technology, and FIG. 1B is an isometric view of the sterilization system having an access door in an open position.

FIGS. 2A and 2B are front and side elevation views, respectively, of the sterilization system of FIGS. 1A and 1B; FIGS. 2C and 2D are side and top across-sectional views, respectively, of the sterilization system; and FIG. 2E is a side isometric view of the sterilization system.

FIG. 3 is a schematic diagram illustrating an electrical system of the sterilization system of FIGS. 1A and 1B configured in accordance with embodiments of the present technology.

FIGS. 4A and 4B are a top, front isometric view and a bottom, front isometric view, respectively, of a UV-C light sterilization system configured in accordance with other embodiments of the present technology; FIG. 4C is a partially-exploded, top, rear isometric view of the sterilization system of FIGS. 4A and 4B with an upper cover removed for clarity; and FIG. 4D is a bottom, front isometric view of the sterilization system of FIGS. 4A and 4B with an access door and a lower cover removed for clarity.

FIG. 5A is an isometric view of a rack configured to be installed within a chamber of the sterilization system of FIGS. 4A-4D in accordance with embodiments of the present technology; and FIG. 5B is a front isometric view of the sterilization system with the rack installed within the chamber.

FIG. 6 is a schematic diagram of an electrical system of the sterilization system of FIGS. 4A and 4B configured in accordance with embodiments of the present technology.

### DETAILED DESCRIPTION

The following disclosure describes various embodiments of systems and methods that use light (e.g., UV-C light) to sterilize items, such as safety goggles, keys, fobs, office supplies, portable electronic devices, respirators, face masks, other personal protection equipment (PPE), and/or a wide variety of other items. In some embodiments, the systems described herein include a cabinet or enclosure that includes a movable support structure (e.g., a rotatable carousel) upon which one or more items can be mounted for sterilization. The enclosure can further include a plurality of UV-C light sources that are positioned so that all, or at least substantially all, of the surfaces of the items are exposed to UV-C light as they move within the enclosure on the support structure. For example, as described in greater detail below, in some embodiments the sterilization systems described herein can include at least one UV-C light source positioned in, or at least near, the center of a rotatable carousel that supports the items for sterilization, and one or more (e.g., four) UV-C light sources arranged around the outside of the carousel. As the carousel rotates, the items mounted on the carousel are thoroughly exposed to the UV-C light, thereby comprehensively sterilizing the items. Accordingly, the embodiments of the technology described herein can move (e.g., rotate) items while irradiating them with UV-C light from within the circle of rotation and from outside the circle of rotation to satisfactorily disinfect the items so that they can be reused.

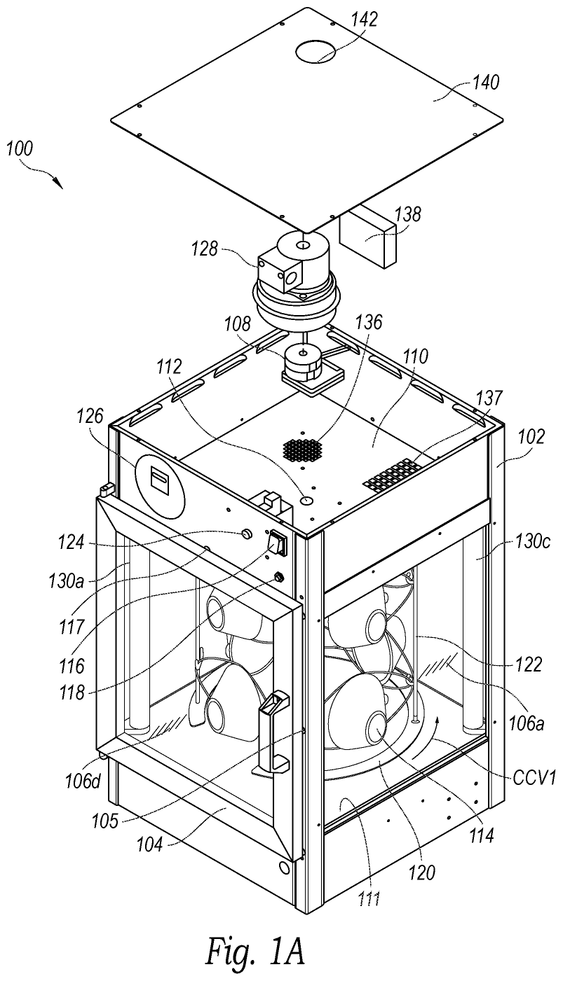

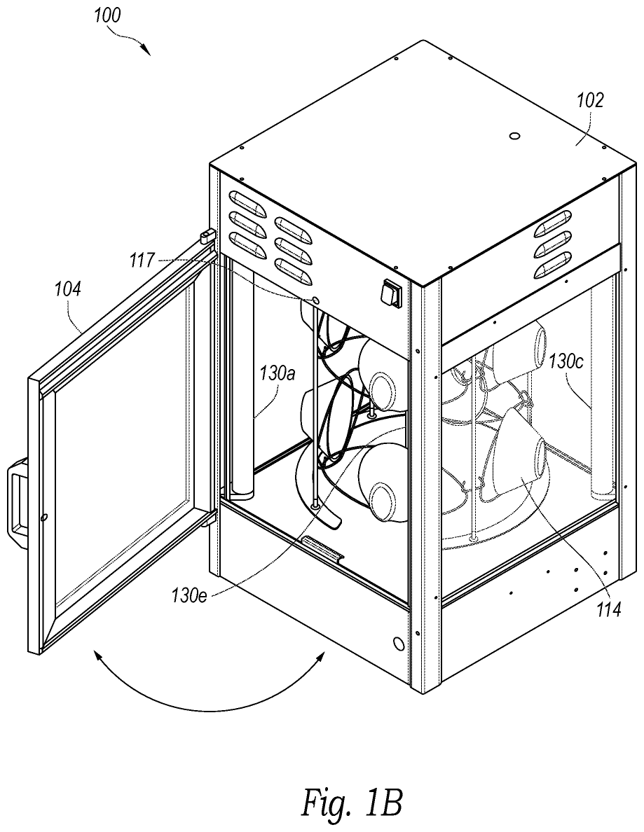

Certain details are set forth in the following description and in FIGS. 1-6 to provide a thorough understanding of various embodiments of the present technology. In other instances, well-known structures, materials, operations and/or systems often associated with UV light sources, UV disinfection equipment, etc., are not shown or described in detail in the following disclosure to avoid unnecessarily obscuring the description of the various embodiments of the technology. Those of ordinary skill in the art will recognize, however, that the present technology can be practiced without one or more of the details set forth herein, or with other structures, methods, components, and so forth.

Several of the embodiments described and illustrated herein are explained by way of example in the context of sterilizing personal protective equipment (PPE), such as respirators. However, the methods and systems described herein can be used to sterilize items other than PPE. Moreover, the descriptions and illustrations of sterilizing PPE are not intended to signify or represent that any related products are functional to sterilize PPE or other medical and/or nonmedical equipment to any relevant medical and/or regulatory standards.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope. The sizes of various depicted elements are not necessarily drawn to scale, and these various elements may be arbitrarily enlarged to improve legibility. Component details may be abstracted in the Figures to exclude details such as position of components and certain precise connections between such components when such details are unnecessary for a complete understanding of how to make and use the invention.

Many of the details, dimensions, angles and other features shown in the Figures are merely illustrative of particular embodiments of the disclosure. Accordingly, other embodiments can have other details, dimensions, angles and features without departing from the spirit or scope of the present invention. In addition, those of ordinary skill in the art will appreciate that further embodiments of the invention can be practiced without several of the details described below.

In the Figures, identical reference numbers identify identical, or at least generally similar, elements. To facilitate the discussion of any particular element, the most significant digit or digits of any reference number refers to the Figure in which that element is first introduced. For example, element 110 is first introduced and discussed with reference to FIG. 1.

FIG. 1A is a partially exploded isometric view of a UV-C light sterilization system 100 configured in accordance with embodiments of the present technology, and FIG. 1B is an isometric view of the UV-C light sterilization system 100 with an access door 104 rotated to an open position. Referring to FIGS. 1A and 1B together, the UV-C light sterilization system 100 (“sterilization system 100”) includes a rotatable support structure 120 (referred to herein as a carousel 120) that is rotatably mounted in a housing 102 (which can also be referred to as a cabinet, enclosure, box, and the like). In the illustrated embodiment, the cabinet 102 is a 4-sided cabinet having transparent (e.g., glass, tempered glass) panels 106a-c affixed to three of the four sides. The access door 104 is hingedly mounted to the fourth side of the cabinet 102 and can also include a transparent panel 106d. In other embodiments, all or some of the transparent panels 106a-d can be omitted. For example, in some embodiments, the three sides of the cabinet 102 that do not include the access door 104 can be completely covered by a non-transparent material, such as sheet metal. In other embodiments, the transparent panel 106d on the access door 104 can be omitted and the access door 104 can be covered by a sheet metal panel or other non-transparent material. In further embodiments, all or some of the four sides of the cabinet 104 can be partially or completely covered by other opaque materials, or materials that are at least opaque to UV-C light. In other embodiments, the sidewalls and/or the door 104 of the cabinet 102 can be formed from other materials including transparent, non-transparent, opaque, and/or partially opaque materials. The framework, cover panels and/or other structure of the cabinet 102 can be formed from a suitable metal material, such as stainless steel (e.g., stainless sheet sheet). In one aspect of this embodiment, the glass of the transparent panels 106a-d and the steel cover panels/framework of the cabinet 102 prevent UV-C from passing through the sidewalls of the cabinet 102.

In the illustrated embodiment, the carousel 120 includes a rack or framework 122 that includes features that facilitate arrangement and attachment of a plurality of items 114 thereon for sterilization within the cabinet 102. In the illustrated embodiment, the items 114 can be respirators (e.g., N95 respirators), but in other embodiments, the items 114 can include other items for sterilization, including other PPE items (e.g., masks, googles, gloves, clothing, etc.) as well as other items. Moreover, although the carousel 120 is configured to carry, for example, eight items 114 in the illustrated example, in other embodiments carrousels and other support structures configured in accordance with the present technology can be configured to carry more or fewer items. Referring to FIG. 1A, the carousel 120 includes a central shaft (not shown) extending upwardly therefrom that is operably coupled to motor 108 (e.g., an electric motor). The motor 108 is mounted to an upper panel 110 of the cabinet 102 such that its drive shaft (not shown) extends through an opening 112 in the upper panel 110 and is coupled to the central shaft of the carousel 120. In this configuration, the carousel 120 is suspended from the motor 108. As described in greater detail below, in operation, the motor 108 rotates the carousel 120 about its central axis in, for example, the counter-clockwise (CCW) direction at a rate of, for example, from about 1 revolution per minute (RPM) to about 30 RPM, from about 2 RPM to about 12 RPM, or about 4-5 RPM. In other embodiments, the motor 108 can rotate the carousel 120 at other speeds and/or in the clockwise (CW) direction, or alternatingly in both the CW and CCW directions for, for example, predetermined periods of time or variable times. In yet other embodiments, the motor 108 can be mounted to a lower panel 111 of the cabinet 102 such that its drive shaft extends upward toward the upper panel 110.

The sterilization system 100 further includes a plurality of UV-C light sources 130 positioned within the cabinet 102. More specifically, in the illustrated embodiment, UV-C light sources 130a-d are individually positioned in each of the four interior corners of the cabinet 102, and a fifth UV-C light source 130e (FIG. 1B) is positioned in the center of the cabinet 102 coaxially aligned, or at least substantially coaxially aligned, with the central axis of the carousel 120. The UV-C light sources 130 can be mounted to the lower panel 111 and/or the upper panel 110 of the cabinet 102. In the illustrated embodiment, the UV-C light sources 130a-e are generally tubular or cylindrically shaped lamps that are vertically oriented and emit light radially outward omnidirectionally, or at least substantially omnidirectionally. In other embodiments, UV-C light sources having other shapes, sizes, positions and/or orientations can be used. The UV-C light sources 130a-e can include, for example, UV-C lamps that provide UV-C light at a wavelength of from about 200 to about 280 nanometers (nm), from about 240 to about 260 nm, or about 254 nm. In one aspect of this embodiment, using UV-C lamps that provide UV light at 254 nm wavelength creates little or no ozone, which could potentially present a potential health risk for operators or persons wearing sterilized items. By way of example, in some embodiments in which the items 114 are respirators, the UV-C light sources 130a-e of the sterilization system 100 can be configured to expose the items 114 to 60 millijoules per centimeter squared (mJ/cm2) of UV-C light to adequately sterilize the items 114. In other embodiments, the sterilization system 100 can be configured to expose items to different levels of UV-C light for sterilization.

In some embodiments, each of the UV-C light sources 130a-e can include a 15-watt bulb configured to emit a wavelength of about 254 nm (e.g., 253.7 nm), with a UV output of 4.9 watts per lamp and operating at 55 volts. An example of such a bulb is the germicidal lamp available from Ushio America, Inc. under the part number G15T8. In other embodiments, other types of commercially available UV-C lamps, bulbs, or light-emitting devices can be used for the UV-C light sources 130a-e, including UV-C light sources operating at different input and output wattages and emitting different wavelengths of UV-C light. Additionally, in some embodiments, all of the light sources 130a-e can be the same type of light source, while in other embodiments some or all of the light sources 130a-e may be different types of light sources.

As shown in FIG. 1A, the sterilization system 100 further includes an exhaust blower 128 and a filter 138 (e.g., a replaceable, activated charcoal filter). The upper panel 110 of the cabinet 102 includes an exhaust outlet 136 and one or more fresh air inlets 137. The exhaust blower 128 is mounted to the upper panel 110 in fluid communication with the exhaust outlet 136. During operation of the sterilization system 100, fresh air flows into the cabinet 102 via the inlet 137 while the exhaust blower 128 sucks exhaust air from within the cabinet 102 (which could potentially include ozone) and drives this exhaust air through the filter 138. After passing through the filter 138, the filtered exhaust air flows out of the cabinet 102 through an opening 142 in a top panel 140. The exhaust blower 128 and the filter 138 enable the sterilization system 100 to be used in an area that may not have a ventilation hood. In some embodiments, the filter 138 can be positioned in the space above the upper panel 110 as illustrated in FIG. 1A. In other embodiments, the filter 138 (or an additional filter) can be positioned upstream of the blower 128 and below the cabinet exhaust outlet 136 (e.g., on the underside of the upper panel 110) so that the filter 138 can be more easily replaced.

In some embodiments, the sterilization system 100 may not include an external exhaust system for exhausting air from the cabinet. In the absence of an external exhaust system, the sterilization system 100 can include a blower or other means to recirculate the exhaust air from the cabinet 102, past the filter 138 and back into the cabinet 102. In other embodiments, such a recirculation system can be used in conjunction with an external exhaust system. The use of a recirculation system could address concerns that the air flow from the cabinet 102 could include pathogens that, even with the filter 138, could potentially flow into an unventilated room or other area in which the sterilization system 100 is located.

In the illustrated embodiment, the sterilization system 100 includes a number of user-operable controls to facilitate user operation of the system. For example, in the illustrated embodiment, the sterilization system 100 includes a main power switch 116, a start cycle button 118, and a cycle timer 124. As described in greater detail below, the main power switch 116 enables the user to activate power to the sterilization system 100, and the cycle timer 124 can include, for example, a rotary knob with which the user can set an appropriate time period for exposing the items 114 to UV-C light for sterilization in the cabinet 102. In other embodiments, other types of manual switches and user interfaces can be provided for the user to set the cycle time. Once the door 104 has been shut, power to the system has been turned on, and the cycle time has been set, the user can initiate the sterilization cycle by depressing the start cycle button 118. This activates a solenoid lock 105 on the door 104. In some embodiments, the start cycle button 118 can illuminate when activated to indicate that the machine is operating. When the unit is operating and the timer 124 is running, the solenoid door lock 105 locks the door 104 so that the operator cannot open the door 104 while the UV-C light sources 130a-e are operating. Since UV-C light can potentially cause skin burns or eye damage, this interlocking door feature prevents an operator from inadvertently injuring themselves. Once the indicator light has gone out, indicating that the cycle is complete, the solenoid door lock 105 is deactivated to enable the user to open the door 104 as shown in FIG. 1B for removal of the sterilized items 114.

To facilitate removal of the items 114, the sterilization system 100 can further include a jog button 117 that is positioned behind the door 104 and thus inaccessible and inoperative while the door 104 is shut. Depressing the jog button 117 incrementally activates the motor 108, which in turn causes the carousel 120 to rotate a pre-set amount (e.g., 45 degrees) about its rotational axis. Repeatedly depressing the jog button 117 enables the operator to sequentially position the items 114 adjacent to the door opening, thereby enabling the operator to easily retrieve them from the interior of the cabinet 102.

The UV light intensity produced by the UV-C light sources 130a-e can deteriorate over time from use. To address this concern, the sterilization system 100 can further include a radiometric data display 126 that is operably connected to a radiometer (not shown in FIGS. 1A and 1B) that is positioned within the interior of the cabinet 102. In operation, the radiometer can detect the level of UV-C light intensity within the cabinet 102, and this information can be displayed (e.g., digitally displayed) to the operator via the radiometric data display (e.g., an LCD) 126. In this manner, the operator can monitor the UV-C light intensity during the sterilization process to ensure that it is maintained at an appropriate level and change out the UV-C light sources 130a-e when the intensity is no longer sufficient.

As noted above, in some embodiments, the sterilization system 100 can include a radiometer to measure the dosage or level of UV light intensity. In other embodiments, less expensive means can be used to measure the UV-C dosage. For example, in some embodiments, adhesive-backed strips that change color to indicate the level of UV-C light intensity can be positioned within the cabinet 102 to provide this information to the operator. If the operator notices that the light intensity is deteriorating, the operator can increase the time setting to achieve the desired overall level of exposure needed to compensate for the deterioration of the lamps. During times of heavy use, the adhesive-backed strips can be changed out daily to ensure that the required exposure is achieved for the timer setting being used. By way of example, the adhesive-backed strips can be obtained from UV Process Supply, Inc., under the brand name Con-Trol-Cure, UV-C intensity labels, P/N N010-004. In other embodiments, other types of adhesive-backed strips and other means for measuring the UV-C light intensity within the cabinet 102 can be used.

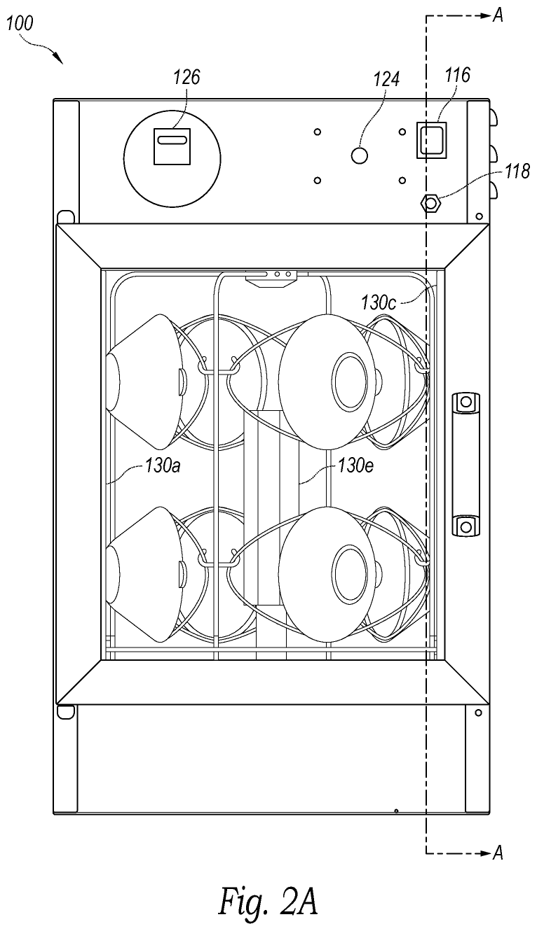

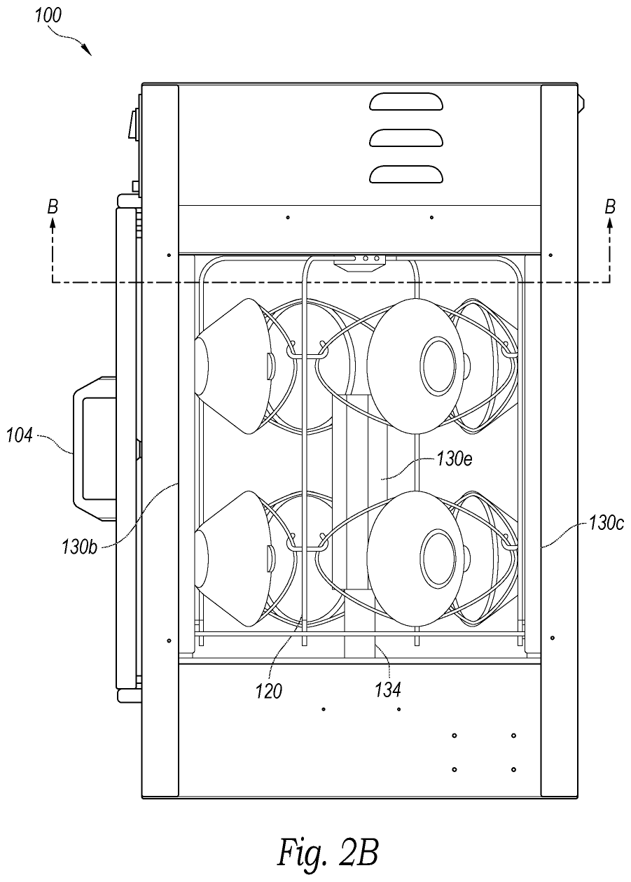

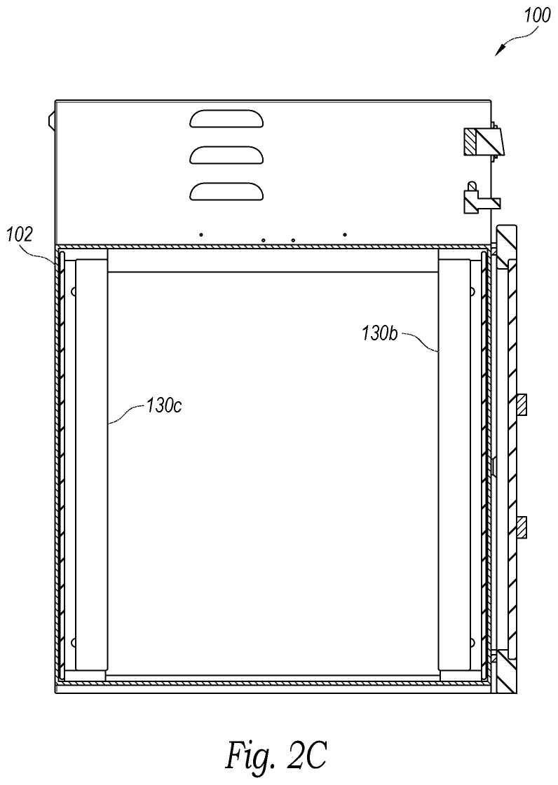

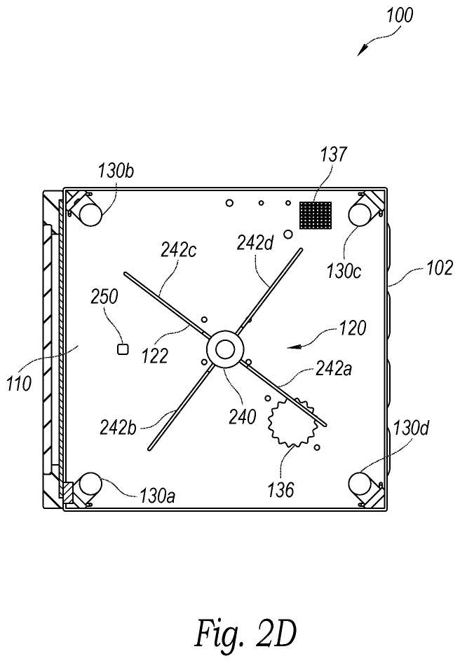

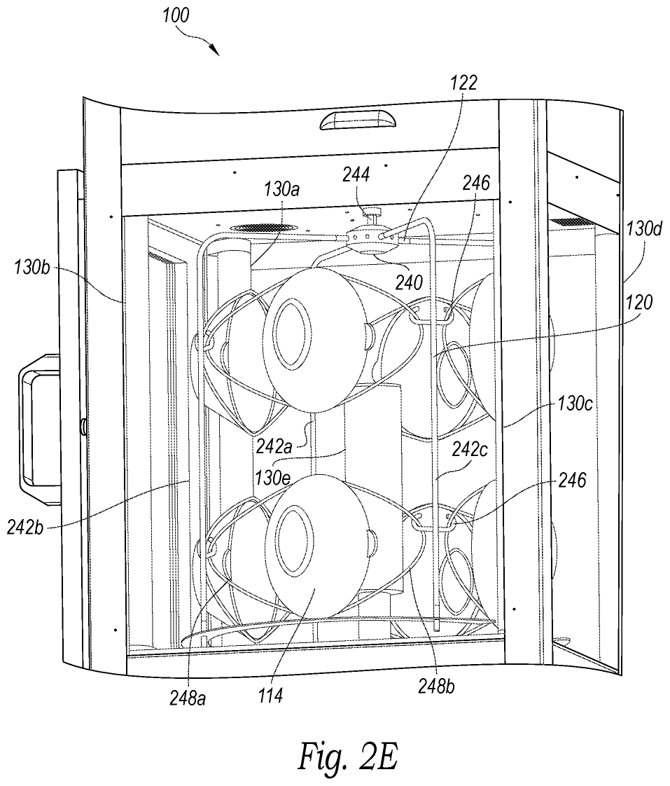

FIGS. 2A and 2B are front- and right-side elevation views, respectively, of the sterilization system 100 configured in accordance with embodiments of the present technology. Referring to FIGS. 2A and 2B together, by way of example the sterilization system 100 can have a width of about 18 inches, a depth of about 18 inches, and a height of about 30 inches so that the unit can be placed and operated on a standard countertop. Additionally, the sterilization system 100 can be light enough so that it can be moved by a single operator. In other embodiments, the sterilization system 100 can have other dimensions, including larger cabinets to accommodate more sterilization items 114, as well as smaller units that might accommodate fewer than the eight sterilization items 114 of the illustrated embodiment.

As shown in FIG. 2B, the central UV-C light source 130e can be positioned proximate a center portion of the cabinet 102 and/or longitudinally aligned with the central axis of rotation of the carousel 120 so that the items 114 rotate around the light source 130e. This enables the light source 130e to irradiate the interior or inward-facing surfaces of the items 114 that cannot be reached by the UV-C light sources 130a-d positioned around the outside of the carousel 120 in the respective corners of the cabinet 102 (which can irradiate the exterior or outward-facing surfaces of the items 114). In the illustrated embodiment, the central light source 130e can be elevated as needed on a stanchion 134. It should be noted that in some embodiments all of the UV-C light sources 130a-e can utilize the same UV-C lamp, but in other embodiments, the light sources may be different. For example, in some embodiments, the light sources arranged around the outside of the carousel 120 can be a first type of UV-C light source, while the central light source 130e can be a different type of UV-C light source. Additionally, as those of ordinary skill in the art will appreciate, in other embodiments, other arrangements of UV-C light sources, as well as other numbers of the UV-C light sources can be used without departing from the present technology. By way of example, in other embodiments, two or more UV-C light sources can be positioned within the carousel 120, while more than four or less than four UV-C can be arranged around the outside of the carousel 120, and or above/below the carrousel 120. Moreover, while a rotating carousel 120 is depicted in the illustrated embodiment, those of ordinary skill in the art will appreciate that other types of moveable support systems, racks, and the like can be used to moveably support the sterilization items 114 during the sterilization process consistent with the present disclosure.

FIG. 2C is a side cross-sectional elevation view of the sterilization system 100 taken along line A-A in FIG. 2A, and FIG. 2D is an upwardly-looking top cross-sectional view of the sterilization system 100 taken along line B-B in FIG. 2B. Turning first to FIG. 2C, this view illustrates that each of the UV-C light sources 130a-d is longitudinally aligned with, and positioned in, a respective interior corner of the cabinet 102 in the illustrated embodiment. Turning next to FIG. 2D, this view further illustrates the placement of the UV-C light sources 130a-d, as well as placement of a radiometer 250, which is mounted to the underside of the cabinet upper panel 110. As described above with reference to FIG. 1A, the radiometer 250 is operably connected to the radiometric data display 126 (FIG. 1A) and configured to provide the operator with information regarding the level of UV light intensity within the cabinet 102 during operation of the sterilization system 100. Also shown to good effect in FIG. 2D is the inlet 137 for introducing fresh air into the cabinet 102 during operation of the sterilization system 100, and the outlet 136 which receives air from within the cabinet 102 during operation of the sterilization system 100 and passes it to the filter 138 by means of the exhaust blower 128 (thus the cabinet outlet 136 is an “inlet” for the exhaust blower; FIG. 1A).

FIG. 2E is an isometric side view illustrating the carousel 120 within the cabinet 102. As this view illustrates, the central hub 240 is fixedly attached to a drive shaft 244 of the motor 108 (FIG. 1A). Accordingly, operation of the motor 108 rotates the drive shaft 244 about its central axis, which in turn rotates the carousel 120 at the desired rotational speed. Referring to FIGS. 2E and 2D together, in the illustrated embodiment, the carousel framework 122 includes a plurality of elongate frame members 242 (e.g., four frame members 242a-d) that extend radially outward from a central hub 240. Each of the frame members 242a-d extend laterally outward from the hub 240 and then downwardly parallel to the sidewalls of the cabinet 102, before terminating just above a floor panel in the interior of the cabinet 102. Additionally, each of the elongate frame members can include a plurality of attachment features (e.g., hooks 246) that are configured to engage portions (e.g., mounting straps 248a, b) of the individual sterilization items 114 and temporarily hold the items 114 in position as the carousel 120 rotates about its central axis. In the illustrated embodiment, the hooks 246 are configured so that the items 114 (e.g., respirators) are favorably positioned with respect to the UV-C radiation for effective sterilization. In the illustrated embodiment, the frame members 242a-d and the hooks 246 can be made from a suitable material, such as steel wire or rod. As will be appreciated by those of ordinary skill in the art, the foregoing is but one example of a suitable framework that can be used to support the items 114 during the sterilization process. Indeed, it is contemplated that numerous other suitable support structures can be configured to support respirators and/or other types of items during the sterilization process.

To properly sterilize items with the sterilization system 100, the items should be exposed to an appropriate level of UV-C radiation (which can also be referred to as dosage energy), such as about 60 mJ/cm2. Dosage energy (mJ/cm2)=UV Intensity (mW/cm2)×Time (seconds). By way of example, in some embodiments the sterilization system 100 can provide a UV intensity of over 1.5 mW/cm2, thereby being capable of providing a dosage energy of 60 mJ/cm2 in less than 1 minute. Some articles will require higher dosage energy and the exposure time will be longer. Generally, the UV Intensity is fixed (although it may diminish somewhat over time), thus the operator can choose the operating time needed to get the dosage energy they deem necessary to treat the article in question. As will be appreciated, the amount of time needed to irradiate respirators and other items 114 for sterilization and the number of times that sterilized respirators can be reused are both parameters that may need to be developed by qualified end-users of the sterilization system 100. By way of example, it is expected that in some embodiments, a treatment of five minutes or less will be sufficient to sterilize most types of respirators and other similar items, and that such items can sustain a minimum of four irradiation sterilization treatments using the sterilization system 100 (equating to five “wearings” or “uses” of the respirators in total).

In the illustrated embodiment, the items 114 are depicted as respirators (or facemasks). Accordingly, as illustrated in FIG. 2E, the carousel 120 of the present embodiment can support eight respirators when driven by the overhead motor 108. Using the UV-C light sources 130a-e described above, it is expected that eight respirators can be properly disinfected and sterilized in four to five minutes. It will be appreciated that these parameters are provided by way of example only, and other items, light sources, rotational speeds, carousels, etc. may require longer or shorter exposure times. Because the carousel 120 rotates during operation, the UV-C light reaches all, or at least substantially all, surfaces of the items 114.

Some research suggests that the structural integrity of respirators diminishes over time and exposure to UV-C light, and suggests that the outside of respirators should receive at least 3.2 times the level of UV-C light exposure that the inside of the respirator is exposed to. Without wishing to be bound by theory, it is contemplated that embodiments of the present technology, which use four outer lamps and one inner lamp, meet this requirement by providing four times the exposure to the outside of the respirator as to the inside.

While the foregoing description has described the use of the sterilization system 100 in the context of respirators, face masks, goggles, and/or other PPE items, it will be appreciated that the systems and methods described herein can be used for sterilization of a wide variety of other items without departing from the present technology. Such items can include, for example, other wearable items, mobile phones and other electronic devices, writing implements, credits cards, currency, and other personal items, products, etc.

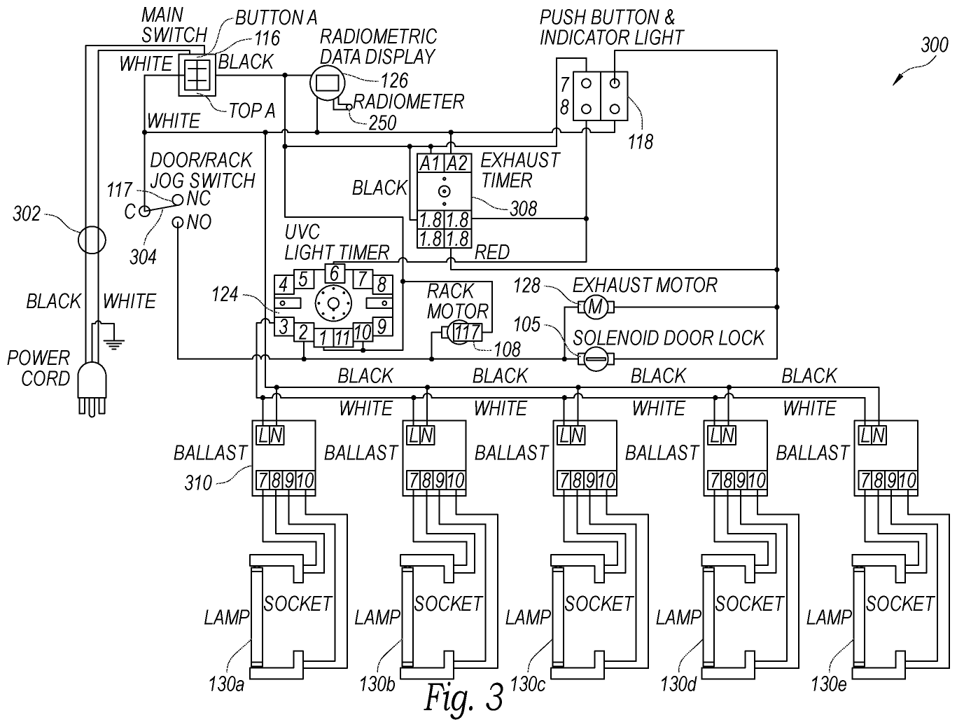

FIG. 3 is a schematic diagram of an electrical system 300 of the sterilization system 100, configured in accordance with embodiments of the present technology. As described above with reference to FIG. 1A, the sterilization system 100 can include a main power switch 116 that is energized with facility power via a cord 302. In some embodiments, the system can be configured to run on 1-phase voltage (e.g., 120v/1-phase/60 Hz or 230v/1-phase/50 Hz). Power from the main power switch 116 is provided to a door switch 304 which is operably coupled to the door 104 (FIG. 1A), which is depicted in the open position in FIG. 3. When the door switch 304 is in the open position (i.e., the door 104 on the cabinet 102 is open), the jog button 117 is energized and can be activated by the user as described above to sequentially rotate the carousel 120 to position the carousel as desired for installation or removal of items 114 for sterilization. Conversely, when the door 104 is closed, the door switch 304 closes, which renders the jog button 117 inactive and instead energizes the solenoid door lock 105. It should be noted that when the door 104 is closed, not only is the jog button 117 inactive, but it is also inaccessible to the operator by virtue of being covered by the frame of the door 104.

When the door switch 304 is closed, the cycle timer 124 is set, and the start cycle button 118 is activated, power is provided to the UV-C light sources 130a-e, to the carousel motor 108, to the exhaust blower 128, and to the radiometer 250 and radiometric data display 126 as described above. The light sources 130a-e will operate for the time period as set on the cycle timer 124. For some types of UV-C lamps (such as the UV-C lamps described above), ballasts 310 may be required. In some embodiments, the exhaust blower 128 can operate for a pre-set period of time as controlled by an exhaust timer 308. The timer 124 and/or other components of the electrical system 300 can include or be operably connected to a suitable controller (e.g., a programmable logic controller (PLC)) and/or other types of processing devices suitable for executing computer-readable instructions or other pre-programmed instructions for controlling operation of the electrical system 300 in accordance with the methods described above. Although specific circuitry is described above, those of ordinary skill in the art will recognize that other circuitry, including other microprocessor-based systems, can also be used to implement various portions of the sterilization system 100 described herein.

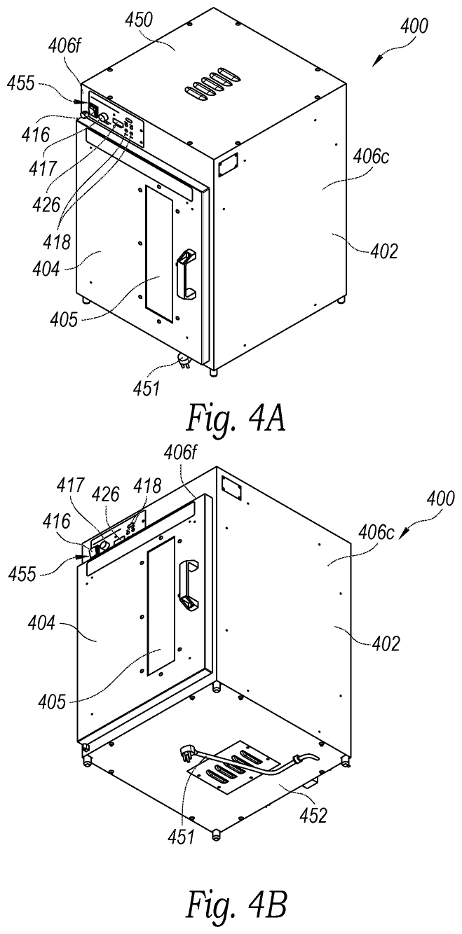

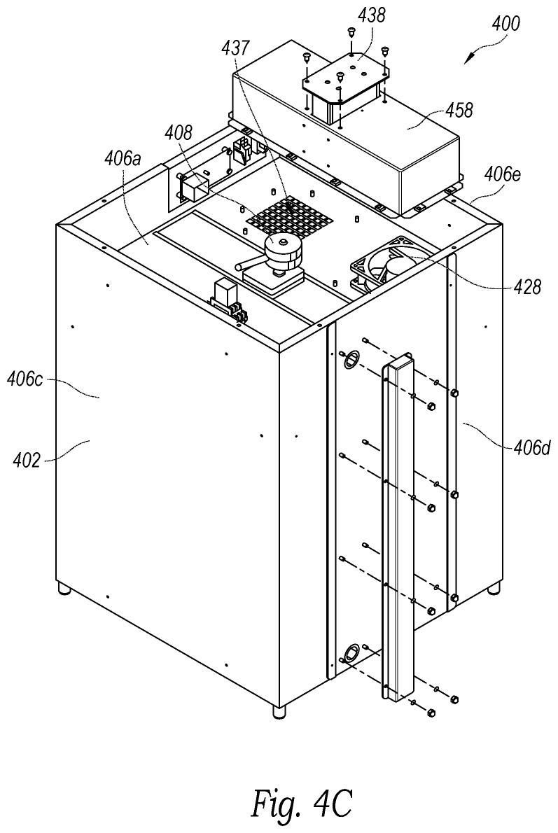

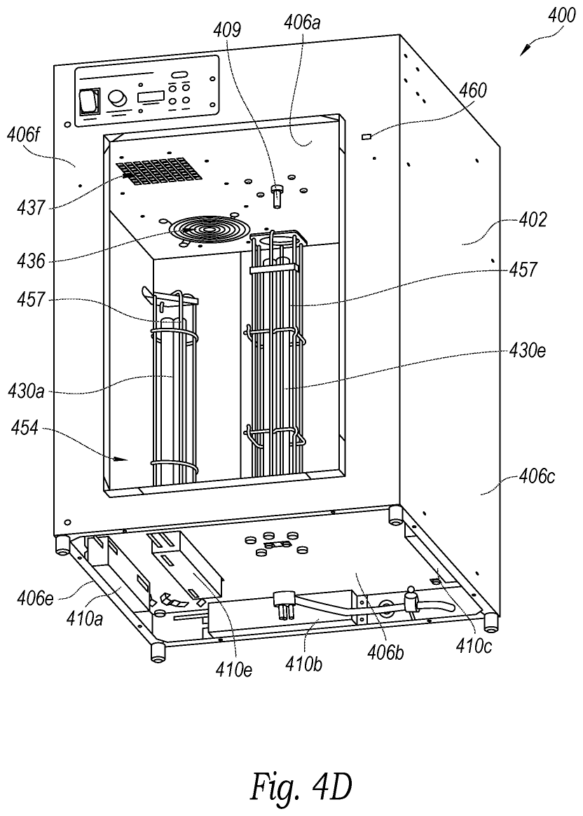

FIGS. 4A and 4B are a top, front isometric view and a bottom, front isometric view, respectively, of a UV-C light sterilization system 400 configured in accordance with additional embodiments of the present technology. The UV-C light sterilization system 400 can include some features that are at least generally similar in structure and function, or identical in structure and function, to the corresponding features of the sterilization system 100 described in detail above with reference to FIGS. 1A-3, and can operate in a generally similar or identical manner to the sterilization system 100. For example, referring to FIGS. 4A and 4B together, the sterilization system 400 can include a housing 402 (e.g., a 4-sided cabinet) and an access door 404 hingedly mounted to the housing 402 along a left vertical edge thereof. The access door 404 is shown in a closed position in FIGS. 4A and 4B. In the illustrated embodiment, the housing 402 includes a top cover or panel 450 and a bottom cover or panel 452. FIG. 4C is a partially-exploded, top, rear isometric view of the sterilization system 400 with the top panel 450 removed for clarity. FIG. 4D is a bottom, front isometric view of the sterilization system 400 with the access door 404 and the bottom panel 452 removed for clarity.

Referring to FIGS. 4A-4D together, the housing 402 further includes a plurality of sides or panels 406 (including an individually identified upper panel 406a, lower panel 406b, and side panels 406c-f) at least partially enclosing an interior volume or 454. The panels 406 can be formed from suitable metal materials, such as stainless-steel sheet. In some embodiments, the panels 406 can be configured to inhibit or even prevent UV-C light from passing therethrough and/or can be configured to reflect UV-C light generated within the chamber 454. In other embodiments, some or all of the panels 406 can be partially or fully transparent to UV-C light, visible light, and/or light of different wavelengths.

Referring to FIGS. 4A and 4B together, in some embodiments the access door 404 includes a window 405. The window 405 can be of glass (e.g., tempered safety glass), plastic, and/or another suitable material and can inhibit or even prevent UV-C light from passing therethrough while permitting at least some visible light to pass therethrough such that, for example, a user can view into the interior volume during operation of the sterilization system 400. In the illustrated embodiment, the sterilization system 400 further includes an electronic control panel 455 coupled to the housing 402 and including a main power switch 416, a jog button 417, a plurality of start cycle buttons 418, and a data display 426. The electronic control panel 455 can be energized with facility power via a cord 451.

Referring to FIG. 4D, the sterilization system 400 further includes a plurality of UV-C light sources 430 positioned within the chamber 454. Similar to the sterilization system 100 described in detail above with reference to FIGS. 1A-3, first through fourth ones of the UV-C light sources 430a-d (the second through fourth UV-C light sources 430b-d are obscured in FIG. 4D) can be individually positioned in each of the four interior corners of the chamber 454, and a fifth one of the UV-C light source 430e (FIG. 1B) can positioned proximate a center of the chamber 454. In some embodiments, the UV-C light sources 430 each include multiple (e.g., two) generally tubular or cylindrically shaped lamps that are vertically oriented and emit light radially outward in, for example, all directions around their peripheries. In other embodiments, the UV-C light sources 430 can include more of fewer lamps and can have other shapes, sizes, positions, and/or orientations. In the illustrated embodiment, the UV-C light sources 430 are each surrounded by a guard 457 (e.g., a cage) configured to protect the UV-C light sources 430 from inadvertent contact that could break or damage the UV-C light sources 430. In some embodiments, each of the UV-C light sources 430a-e includes a corresponding ballast 410a-e that can be secured to the housing 402 (e.g., to the lower panel 406b and/or the side panels 406c-f) in a space below the lower panel 406b and above the bottom panel 452 (FIG. 4B). In other embodiments, one or more UV-C light sources (not shown) can be mounted to the upper panel 406a and/or the floor panel 406b of the sterilization system 400.

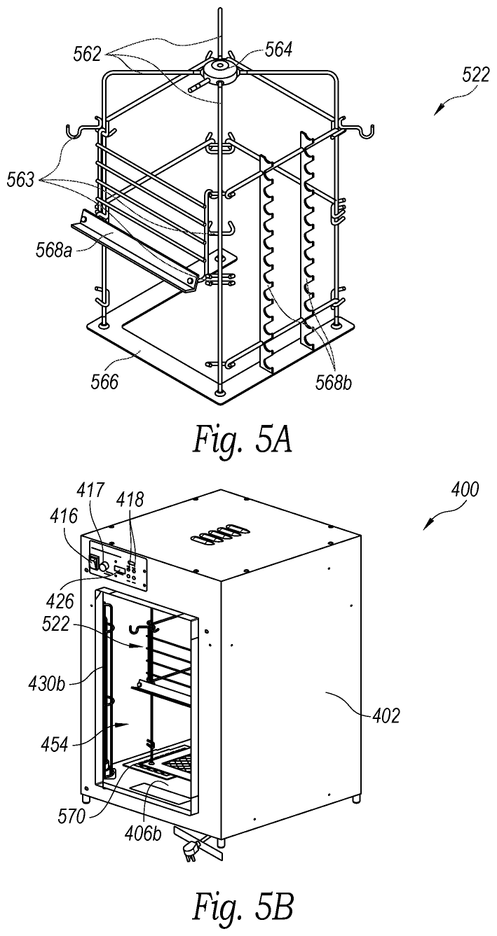

Referring to FIGS. 4C and 4D together, the sterilization system 400 further includes a motor 408 coupled to the upper panel 406a and positioned in a space between the upper panel 406a and the top panel 450 (FIG. 4A). In some embodiments, the motor 408 can include a drive shaft 409 that extends through the upper panel 406a into the chamber 454. As described in greater detail below with reference to FIGS. 5A and 5B, the drive shaft 409 is configured to be coupled to a rack that includes features that facilitate the arrangement and attachment of a plurality of items thereon for sterilization within the chamber 454. In the illustrated embodiment, the upper panel 406a further includes an exhaust outlet 436 and a filtered air inlet 437. An exhaust blower 428 can be mounted to the upper panel 406a in fluid communication with the exhaust outlet 436 and an air duct 458. The air duct 458 fluidly connects the exhaust blower 428 to the filtered air inlet 437 and can include a removable filter assembly 438. During operation of the sterilization system 400, the exhaust blower 428 sucks air from within the chamber 454 (which could potentially include ozone) and drives the air through the air duct 458, through/pass the filter assembly 438, and through the filtered air inlet 437 back into the chamber 454. Accordingly, the sterilization system 400 is configured to recirculate and filter air from within the chamber 454.

Referring to FIGS. 4A, 4B, and 4D together, the sterilization system 400 can include a door switch 460 (FIG. 4D) configured to interact with the access door 404. For example, in some embodiments the door switch 460 can be a mechanical contact switch that is in (i) a closed position when the access door 404 is closed and (ii) an open position when the access door 404 is open. In other embodiments, the door switch 460 can be a magnetic switch, a Hall-effect switch, an optical switch, or another suitable type of switch. When the door switch 460 is in the closed position with the access door 404 closed, the door switch 460 completes an electrical circuit such that the main power switch 416 can be activated to provide power from the cord 451 to the UV-C light sources 430. Conversely, when the door switch 460 is in the open position with the access door 404 open, the door switch 460 can break the electrical circuit such that the main power switch 416 cannot be activated to provide power from the cord 451 to the UV-C light sources 430. Accordingly, in some aspects of the present technology the door switch 460 prevents the UV-C light sources 430 from being turned on when the access door 404 is open. That is, for example, opening the access door 404 during operation of the sterilization system 400 can automatically deenergize the UV-C light sources 430. In some embodiments, the door switch 460 can be configured (e.g., sized, shaped, positioned) such that it is difficult or impossible for a user to manually close the door switch 460 when the access door 404 is in the open position. For example, the door switch 460 can be positioned in a recess is small enough to prevent a user's finger from entering the recess to engage the door switch 460. In some such embodiments, the access door 404 can include an engagement feature (not shown; e.g., a protrusion) sized, shaped, and/or positioned to enter the recess and engage the door switch 460 when the access door 404 is closed. In some embodiments, the access door 404 can further include a latch configured to secure the access door 404 in the closed position.

FIG. 5A is an isometric view of a rack 522 configured to be installed within the chamber 454 of the sterilization system 400 in accordance with embodiments of the present technology. FIG. 5B is a front isometric view of the sterilization system 400 with the rack 522 installed within the chamber 454. The access door 404 is omitted in FIG. 5B for clarity. The rack 522 includes features that facilitate the arrangement and attachment of a plurality of items thereon for sterilization within the chamber 454. More specifically, referring to FIG. 5A, the rack 522 can include a framework 562 (e.g., comprising a plurality of elongate members) extending between a hub 564 and a base 556. In some embodiments, the framework 562 can include a plurality of attachment features 563 (e.g., hooks, loops, protrusions, supports) extending therefrom. The rack 522 can further include a plurality of item supports 568 (e.g., including an individually identified first item support 568a and second item support 568b) coupled to a corresponding one or more of the attachment features 563. The item supports 568 are configured (e.g., shaped sized, positioned) to receive and support one or more items for sterilization. For example, in the illustrated embodiment the first item support 568a includes a platform for receiving and securing a portable electronic device (e.g., a laptop, tablet, mobile phone, and the like), and the second item support 568b includes a plurality of receiving slots for receiving and securing various elongate items (e.g., office supplies, pens, markers, pencils, tools, and the like). In some embodiments, the item supports 568 are removable from the framework 562 and/or interchangeable to allow for customization of the rack 522 depending on the various items a user wishes to sterilize. In other embodiments, the item supports 568 can be permanently attached to or integrally formed with the framework 562. In some embodiments, items for sterilization can be directly mounted to and supported by the framework 562. For example, glasses, keys, fobs, goggles, and/or other items can be directly supported by one or more of the attachment features 563. The rack 522 can be made of metal (e.g., stainless-steel), plastic, and/or other suitably strong and rigid materials.

Referring to FIGS. 4A-5B together, the hub 564 of the rack 522 is configured to be operably coupled to the drive shaft 409 of the motor 408 such that the rack 522 is suspended within the chamber 454 (e.g., above the lower panel 406b). The motor 408 is operable to rotate the rack 522 within the chamber 454. When the rack 522 is installed within the chamber 454, the fifth UV-C light source 430e is positioned within (e.g., radially inside) the rack 522, and the first through fourth UV-C light sources 430a-d are positioned outside (e.g., radially outside) of the rack 522. That is, the fifth UV-C light source 430e is within a circle of rotation of the rack 522 while the first through fourth UV-C light sources 430a-d are positioned radially outward of the fifth UV-C light source 430e outside of the circle of rotation.

To operate the sterilization system 400, a user can first position various items for sterilization on the rack 522. In some embodiments, to facilitate positioning of the items on the rack 522, the user can depress the jog button 417 to drive the motor 408 to sequentially rotate the rack 522 within the chamber 454 to position the rack 522 as desired for installation of items for sterilization. After positioning the items for sterilization, the user can close the access door 404 to seal the chamber 454 and close the door switch 460. Closing of the door switch 460 energizes the main power switch 416 and the start cycle buttons 418. After depressing the main power switch 416, the user can select one of the start cycle buttons 418 to energize the UV-C light sources 430 for a predetermined disinfecting cycle (e.g., one minute, two minutes, five minutes, or more). During the disinfecting cycle, the motor 408 drives the rack 522 to rotate within the chamber 454 while the UV-C light sources 430 illuminate the items thereon. Because the rack 422 rotates during operation, the UV-C light reaches all, or at least substantially all, surfaces of the items. Additionally, the data display 426 can display a time corresponding to the time remaining or elapsed in the disinfection cycle, and/or other data related to the disinfecting cycle. After the disinfecting cycle is over, the user can open the access door and remove the items from the rack 522.

Referring to FIG. 5B, in some embodiments the sterilization system 400 can further include a sheet of UV-C intensity labels 570 affixed to the lower panel 406b or another portion of the housing 402 within the chamber 454. The UV-C intensity labels 570 can provide an indication of the level of UV-C light within the chamber 454 to, for example, ensure that a required exposure is achieved for each timer setting that is used.

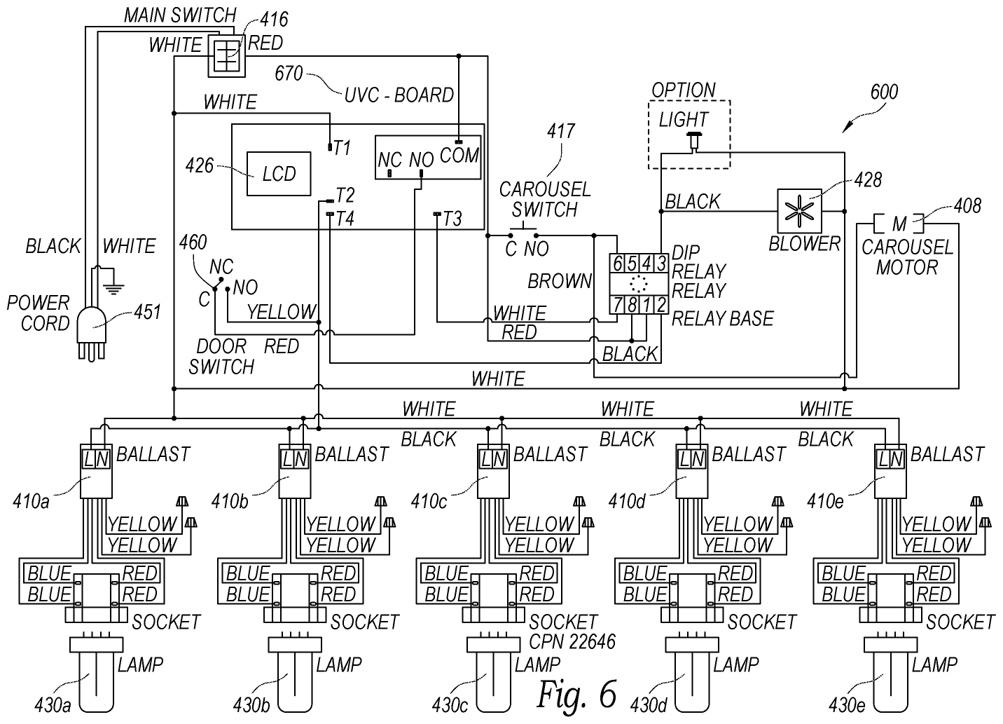

FIG. 6 is a schematic diagram of an electrical system 600 of the sterilization system 400 configured in accordance with embodiments of the present technology. The electrical system can include some features that are at least generally similar in structure and function, or identical in structure and function, to the corresponding features of the electrical system 300 described in detail above with reference to FIG. 3, and can operate in a generally similar or identical manner to the electrical system 300. For example, the electrical system 600 includes the main power switch 416 that is energized with facility power via a cord 451. Power from the main power switch 416 is provided to a door switch 460 which is operably coupled to the door 404 (FIGS. 4A and 4B), which is depicted in the open position in FIG. 6. In some embodiments, one or more components of the electrical system 600 is can be carried by and/or implemented in a programmed logic board 670.

The following examples are illustrative of several embodiments of the present technology:

1. A sterilization system, comprising:
a cabinet at least partially enclosing a chamber;
a support structure positioned within the chamber and configured to move one or more items placed thereon; and
a UV-C light source positioned within the chamber and configured to emit UV-C light for radiating the one or more items as they move within the chamber.

2. The sterilization system of example 1, further comprising a driver operably coupled to the support structure and configured to move the support structure within the chamber relative to the one or more UV-C light sources.

3. The sterilization system of example 1 or example 2, further comprising a driver operably coupled to the support structure and configured to rotate the support structure within the chamber relative to the one or more UV-C light sources.

4. The sterilization system of example 3 wherein the driver is a motor is positioned in a top portion of the cabinet, and wherein the support structure is suspended from the driver within the chamber.

5. The sterilization system of any one of examples 1-4 wherein the UV-C light source is positioned proximate a center of the chamber.

6. The sterilization system of any one of examples 1-5 wherein the UV-C light source is positioned proximate a rotational axis of the support structure.

7. The sterilization system of any one of examples 1-6 wherein the UV-C light source is a first UV-C light source positioned proximate a center of the cabinet, and further comprising a plurality of second UV-C light sources positioned proximate a perimeter of the chamber.

8. The sterilization system of any one of examples 1-7 wherein the support structure defines an interior space, and wherein the UV-C light source is positioned within the interior space defined by the support structure.

9. The sterilization system of example 8 wherein the UV-C light source is a first UV-C light source, and further comprising at least one second UV-C light source positioned within the chamber and outward of the support structure.

10. The sterilization system of example 8 or example 9 wherein the UV-C light source is a first UV-C light source, wherein the chamber includes a plurality of corners, and further comprising a plurality of second UV-C light sources positioned within the chamber in corresponding ones of the corners.

11. The sterilization system of any one of examples 1-10 wherein the UV-C light source includes one or more vertically-oriented elongate UV-C lamps.

12. The sterilization system of any one of examples 1-11 wherein the cabinet includes a chamber inlet and a chamber outlet, and further comprising:
a blower in fluid communication with the chamber outlet;
an air duct fluidly connecting the blower to the chamber inlet; and
a filter positioned along the air duct, wherein the blower is configured to (a) draw air from the chamber through the chamber outlet and (b) blow the air through the air duct past the filter to the chamber inlet.

13. The sterilization system of any one of examples 1-12, further comprising:
an access door operably coupled to the cabinet and movable between an open position that provides access to the chamber and a closed position that prevents access to the chamber; and
a switch operably mounted to at least one of the access door or the cabinet, wherein the switch is configured to respond to movement of the door to the closed position by enabling electrical power to be provided to the UV-C light source, and wherein the switch is further configured to respond to movement of the door away from the closed position by preventing electrical power from being provided to the UV-C light source.

14. The sterilization system of any one of examples 1-13 further comprising:
a driver operably coupled to the support structure and configured to move the support structure within the chamber relative to the one or more UV-C light sources;
an access door hingedly coupled to the cabinet and movable between an open position that provides access to the chamber and a closed position that prevents access to the chamber; and
a jog button operably coupled to the driver, wherein the jog button is operable to actuate the driver to move the support structure when the access door is in the open position.

15. The sterilization system of example 14 wherein the jog button is operable to actuate the driver to rotate the support structure when the access door is in the open position.

16. A sterilization system, comprising:
a cabinet at least partially enclosing a chamber;
a support structure positioned within the chamber and configured to support one or more items for sterilization;
a first UV-C light source positioned within the chamber proximate a center of the chamber, wherein the first UV-C light source is configured to irradiate inwardly-facing surfaces of the one or more items with UV-C light; and
a plurality of second UV-C light sources positioned within the chamber radially outward of the first UV-C light source, wherein the second UV-C light sources are configured to irradiate outwardly-facing surfaces of the one or more items with UV-C light.

17. The sterilization system of example 16 wherein the support structure is rotatable about the first UV-C light source.

18. The sterilization system of example 16 or example 17 wherein the chamber includes a floor panel and a plurality of corners, wherein the first and second UV-C light sources extend upwardly from the floor panel, wherein the second UV-C light sources are positioned in corresponding ones of the corners, and wherein the support structure is suspended above the floor panel.

19. The sterilization system of any one of examples 16-18 wherein the cabinet includes a floor panel, wherein each of the first and second UV-C light sources includes a lamp and a ballast operably coupled to the lamp, wherein each of the lamps is mounted to the floor panel within the chamber, and wherein each of the ballasts is mounted to the cabinet below the floor panel.

20. A method of sterilizing an item, the method comprising:
supporting the item on a support structure within a chamber;
moving the support structure within the chamber; and
while moving the support structure, irradiating the item with UV-C light.

21. The method of example 20 wherein moving the support structure includes rotating the support structure.

22. The method of example 20 or example 21 wherein the support structure defines an interior space, and wherein irradiating the item with UV-C light includes—
energizing a first UV-C light source positioned within the chamber within the interior space of the support structure to irradiate at least an inward-facing surface of the item; and
energizing a plurality of second UV-C light sources positioned within the chamber outside of the interior space of the support structure to irradiate at least an outward-facing surface of the item.

23. The method of any one of examples 20-23 wherein irradiating the item with UV-C light includes energizing a UV-C light source positioned within the chamber, and wherein moving the support structure includes rotating the support structure about the UV-C light source.

References throughout the foregoing description to features, advantages, or similar language do not imply that all the features and advantages that may be realized with the present technology should be or are in any single embodiment of the invention. Rather, language referring to the features and advantages is understood to mean that a specific feature, advantage, or characteristic described in connection with an embodiment is included in at least one embodiment of the present technology. Thus, discussion of the features and advantages, and similar language, throughout this specification may, but do not necessarily, refer to the same embodiment.

Furthermore, the described features, advantages, and characteristics of the present technology may be combined in any suitable manner in one or more embodiments. One skilled in the relevant art will recognize that the present technology can be practiced without one or more of the specific features or advantages of a particular embodiment. In other instances, additional features and advantages may be recognized in certain embodiments that may not be present in all embodiments of the present technology.

Any patents and applications and other references noted above, including any that may be listed in accompanying filing papers, are incorporated herein by reference in the entirety, except for any subject matter disclaimers or disavowals, and except to the extent that the incorporated material is inconsistent with the express disclosure herein, in which case the language in this disclosure controls. Aspects of the invention can be modified, if necessary, to employ the systems, functions, and concepts of the various references described above to provide yet further implementations of the invention.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above Detailed Description of examples and embodiments of the invention is not intended to be exhaustive or to limit the invention to the precise form disclosed above. While specific examples for the invention are described above for illustrative purposes, various equivalent modifications are possible within the scope of the invention, as those skilled in the relevant art will recognize.

The teachings of the invention provided herein can be applied to other systems, not necessarily the system described above. The elements and acts of the various examples described above can be combined to provide further implementations of the invention. Some alternative implementations of the invention may include not only additional elements to those implementations noted above, but also may include fewer elements. Further any specific numbers noted herein are only examples: alternative implementations may employ differing values or ranges.

While the above description describes various embodiments of the invention and the best mode contemplated, regardless how detailed the above text, the invention can be practiced in many ways. Details of the system may vary considerably in its specific implementation, while still being encompassed by the present disclosure. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed in the specification, unless the above Detailed Description section explicitly defines such terms. Accordingly, the actual scope of the invention encompasses not only the disclosed examples, but also all equivalent ways of practicing or implementing the invention under the claims.

From the foregoing, it will be appreciated that specific embodiments of the invention have been described herein for purposes of illustration, but that various modifications may be made without deviating from the spirit and scope of the various embodiments of the invention. Further, while various advantages associated with certain embodiments of the invention have been described above in the context of those embodiments, other embodiments may also exhibit such advantages, and not all embodiments need necessarily exhibit such advantages to fall within the scope of the invention. Accordingly, the invention is not limited, except as by the appended claims.

Although certain aspects of the invention are presented below in certain claim forms, the applicant contemplates the various aspects of the invention in any number of claim forms. Accordingly, the applicant reserves the right to pursue additional claims after filing this application to pursue such additional claim forms, in either this application or in a continuing application.

I/We claim:

1. A sterilization system, comprising:
a cabinet at least partially enclosing a chamber;
a support structure positioned within the chamber and configured to move one or more items placed thereon; and
a UV-C light source positioned within the chamber and configured to emit UV-C light for radiating the one or more items as they move within the chamber.

2. The sterilization system of claim 1, further comprising a driver operably coupled to the support structure and configured to move the support structure within the chamber relative to the one or more UV-C light sources.

3. The sterilization system of claim 1, further comprising a driver operably coupled to the support structure and configured to rotate the support structure within the chamber relative to the one or more UV-C light sources.

4. The sterilization system of claim 3 wherein the driver is a motor is positioned in a top portion of the cabinet, and wherein the support structure is suspended from the driver within the chamber.

5. The sterilization system of claim 1 wherein the UV-C light source is positioned proximate a center of the chamber.

6. The sterilization system of claim 1 wherein the UV-C light source is positioned proximate a rotational axis of the support structure.

7. The sterilization system of claim 1 wherein the UV-C light source is a first UV-C light source positioned proximate a center of the cabinet, and further comprising a plurality of second UV-C light sources positioned proximate a perimeter of the chamber.

8. The sterilization system of claim 1 wherein the support structure defines an interior space, and wherein the UV-C light source is positioned within the interior space defined by the support structure.

9. The sterilization system of claim 8 wherein the UV-C light source is a first UV-C light source, and further comprising at least one second UV-C light source positioned within the chamber and outward of the support structure.

10. The sterilization system of claim 8 wherein the UV-C light source is a first UV-C light source, wherein the chamber includes a plurality of corners, and further comprising a plurality of second UV-C light sources positioned within the chamber in corresponding ones of the corners.

11. The sterilization system of claim 1 wherein the UV-C light source includes one or more vertically-oriented elongate UV-C lamps.

12. The sterilization system of claim 1 wherein the cabinet includes a chamber inlet and a chamber outlet, and further comprising:
a blower in fluid communication with the chamber outlet;
an air duct fluidly connecting the blower to the chamber inlet; and
a filter positioned along the air duct, wherein the blower is configured to (a) draw air from the chamber through the chamber outlet and (b) blow the air through the air duct past the filter to the chamber inlet.

13. The sterilization system of claim 1, further comprising:
an access door operably coupled to the cabinet and movable between an open position that provides access to the chamber and a closed position that prevents access to the chamber; and
a switch operably mounted to at least one of the access door or the cabinet, wherein the switch is configured to respond to movement of the door to the closed position by enabling electrical power to be provided to the UV-C light source, and wherein the switch is further configured to respond to movement of the door away from the closed position by preventing electrical power from being provided to the UV-C light source.

14. The sterilization system of claim 1, further comprising:
a driver operably coupled to the support structure and configured to move the support structure within the chamber relative to the one or more UV-C light sources;
an access door hingedly coupled to the cabinet and movable between an open position that provides access to the chamber and a closed position that prevents access to the chamber; and
a jog button operably coupled to the driver, wherein the jog button is operable to actuate the driver to move the support structure when the access door is in the open position.

15. The sterilization system of claim 14 wherein the jog button is operable to actuate the driver to rotate the support structure when the access door is in the open position.

16. A sterilization system, comprising:
a cabinet at least partially enclosing a chamber;
a support structure positioned within the chamber and configured to support one or more items for sterilization;
a first UV-C light source positioned within the chamber proximate a center of the chamber, wherein the first UV-C light source is configured to irradiate inwardly-facing surfaces of the one or more items with UV-C light; and
a plurality of second UV-C light sources positioned within the chamber radially outward of the first UV-C light source, wherein the second UV-C light sources are configured to irradiate outwardly-facing surfaces of the one or more items with UV-C light.

17. The sterilization system of claim 16 wherein the support structure is rotatable about the first UV-C light source.

18. The sterilization system of claim 16 wherein the chamber includes a floor panel and a plurality of corners, wherein the first and second UV-C light sources extend upwardly from the floor panel, wherein the second UV-C light sources are positioned in corresponding ones of the corners, and wherein the support structure is suspended above the floor panel.

19. The sterilization system of claim 16 wherein the cabinet includes a floor panel, wherein each of the first and second UV-C light sources includes a lamp and a ballast operably coupled to the lamp, wherein each of the lamps is mounted to the floor panel within the chamber, and wherein each of the ballasts is mounted to the cabinet below the floor panel.

20. A method of sterilizing an item, the method comprising:
supporting the item on a support structure within a chamber;
moving the support structure within the chamber; and
while moving the support structure, irradiating the item with UV-C light.

21. The method of claim 20 wherein moving the support structure includes rotating the support structure.

22. The method of claim 20 wherein the support structure defines an interior space, and wherein irradiating the item with UV-C light includes—
energizing a first UV-C light source positioned within the chamber within the interior space of the support structure to irradiate at least an inward-facing surface of the item; and
energizing a plurality of second UV-C light sources positioned within the chamber outside of the interior space of the support structure to irradiate at least an outward-facing surface of the item.

23. The method of claim 20 wherein irradiating the item with UV-C light includes energizing a UV-C light source positioned within the chamber, and wherein moving the support structure includes rotating the support structure about the UV-C light source.
